(19) United States
(12) Patent Application Publication
(10) Pub. No.: US 2021/0361371 A1
(43) Pub. Date: Nov. 25, 2021

(54) FLEXIBLE ISOLATION SYSTEM AND METHOD OF USE

(71) Applicant: Prep Tech, L.L.C., Westlake, LA (US)

(72) Inventors: Mark R. Moore, Westlake, LA (US); Peter Prados, Lafayette, LA (US)

(73) Assignee: Prep Tech, L.L.C., Westlake, LA (US)

(21) Appl. No.: 17/301,698

(22) Filed: Apr. 12, 2021

Related U.S. Application Data
(63) Continuation-in-part of application No. 17/301,217, filed on Mar. 29, 2021.
(60) Provisional application No. 63/001,379, filed on Mar. 29, 2020.
(60) Provisional application No. 62/704,092, filed on Apr. 21, 2020.
(60) Provisional application No. 63/008,331, filed on Apr. 10, 2020.
(60) Provisional application No. 62/704,093, filed on Apr. 21, 2020.
(60) Provisional application No. 62/706,393, filed on Aug. 13, 2020.

(51) Int. Cl. A61B 46/23; A61B 17/132

(52) U.S. Cl. CPC A61B 46/23; A61B 2017/00902; A61B 17/1322

(57) ABSTRACT

An isolette comprising an array of support structures is provided, covered by sheeting, and a drape. The arrays provide vertical or peripheral wire frame supports which are easily assembled and attached to the flexible sheeting. Access ports allow patient access. A component access panel is provided. The drape includes an optional sealing ring held in position by a strap or medical tape. The access ports can include iris diaphragms, covering flaps, integrated gloves, and removable gloves.

### CROSS-REFERENCE TO RELATED APPLICATIONS

This application is a continuation-in-part of U.S. application Ser. No. 17/301,217, filed on Mar. 29, 2021, which claims priority benefit from U.S. Provisional Application No. 63/001,379, filed on Mar. 29, 2020. This application claims priority benefit from U.S. Provisional Application No. 63/008,331, filed on Apr. 10, 2020, U.S. Provisional Application No. 62/704,093, filed on Apr. 21, 2020 and U.S. Provisional Application No. 62/706,393 filed on Aug. 13, 2020. The patent applications identified above are incorporated herein by reference in their entirety to provide continuity of disclosure.

### FIELD OF THE INVENTION

The present invention relates to medical isolettes.

### BACKGROUND OF THE INVENTION

Patient isolation represents one of several measures that can be taken to control the spread of infection and highly contagious diseases. The goal of patient isolation is to contain airborne contagions produced by the patient, or alternatively, to limit airborne contagions to which the patient is exposed.

The U.S. Centers for Disease Control and Prevention (CDC) issues, and periodically revises, recommendations for various levels of patient isolation for disease control. Isolation is typically recommended when a patient is known to have a highly contagious viral or bacterial illness.

Special isolation protocols are used in the management of patients in isolation. Typical isolation protocols include mandating personal protective equipment such as gowns, masks, and gloves and applying physical controls, such as negative pressure rooms, laminar air flow and structural barriers. Negative pressure is used to reduce risk of exposure to airborne contagions produced by the patient.

Patients can produce airborne contagions in any number of circumstances. For example, airborne contagions are typically produced by a patient during intubation. Intubation is required when the patient cannot maintain their airway or cannot breathe without assistance. Intubation requires inserting an endotracheal tube, through the mouth and then into the airway. The tube is then connected to a ventilator. Intubation typically takes place after a patient is anaesthetized. Even so, patient reflex reactions usually cause coughing during the intubation procedure which can result in a significant amount of airborne contagion being released.

The requirement for patient isolation and intubation occurs in many circumstances. For example, patient isolation and intubation can take place in an emergency situation in the field. Emergency isolation in the field is difficult because physical control protocols such as negative pressure rooms and structural barriers are difficult to erect quickly and at low cost. Further exacerbating the problem, intubation is typically prescribed only once a dangerous condition is recognized. Oftentimes, such diagnoses are made in emergencies with extremely limited time to react.

Emergency situations, such as epidemics, also create special problems. For example, epidemics, such as swine flu, MERS-COV, Ebola and COVID-19 are considered highly contagious. Such highly contagious diseases certainly require effective negative pressure isolation and many times also require patient intubation, simultaneously. Effective negative pressure containment is critical to control rapid transmission of these diseases. In these situations, it is extremely difficult to protect caregivers during the intubation process, given the current state of the art in isolation barriers.

The prior art has attempted many different methods and structures to solve the problems of simple, inexpensive and portable isolation barriers but all have fallen short.

For example, U.S. Pat. No. 5,832,919 issued to Kano discloses a portable enclosure system which provides a suitable breathing atmosphere within a chamber, by use of positive pressure provided by fans and exhaust ports. However, the chamber does not allow access to the patient to provide medical treatment and fails to provide a negative pressure environment.

U.S. Pat. No. 7,757,689 issued to Chang discloses an inflatable isolation “cabin” for isolation of the entire patient, prior to transport. However, the cabin must be removed to provide medical treatment such as surgery or intubation.

Another example is disclosed in U.S. Pat. No. 4,949,714, to Orr. Orr discloses a medical hood for fitting over the head of a patient. A gas port leads to the interior of the hood for supplying respiratory gas. An outlet connects the interior of the hood and the ambient air for discharge of gas. However, the hood of Orr is rigid and therefore does not provide for a collapsible frame for ease of use and transport.

Another example is U.S. Pat. No. 5,950,625 to Bongiovanni, et al. Bongiovanni discloses an isolation bag for isolating a casualty from a contaminated environment. The bag is fabricated from a transparent biochemically resistive material and includes a self-contained transportable life support system. However, the transportable system, including the bag, is difficult to replicate cheaply or transport easily and is time consuming to assemble and use.

A need exists to provide a device and method to isolate a patient while allowing the patient to be an intubated or receive other medical treatment. Further, a need exists for an isolation chamber that may be quickly deployed to protect health care providers from contagions while completing the intubation procedure. Still further, a need exists for an isolette that is inexpensive and which can be easily and quickly mass produced to aid in mass isolation of patients with highly contagious epidemic diseases.

A further need exists for an isolette that provides a negative pressure environment to protect health care workers when accessing a patient.

### SUMMARY OF THE INVENTION

A preferred embodiment includes a rigid base supporting a set of vertical stanchions. The stanchions support a flexible enclosure and a patient drape sufficient to isolate the head and upper torso of a patient. The flexible enclosure includes two side panels, a front panel and a top panel. Each side panel may include one or more access ports with integrated or removable bi-directional gloves. The front panel is sized to accommodate two side-by-side access ports which may include integrated or removable bi-directional gloves. A pressurization port is provided for connection to a turbine negative pressure unit. A component access panel is provided which serves as a pass through for ventilation tubes and patient support leads.

In a preferred embodiment, the side walls and front wall can further include resealable flaps, sealing caps or iris diaphragms that cover and seal the access ports.

In one preferred embodiment, the component access panel is inductively welded into the side wall and includes removable sealed plugs of various sizes. The plugs accommodate various sizes of tubes and electrical leads.

In another preferred embodiment, the component access panel is provided with various integrated connectors for connection to a patient circuit and Luer Lock or other connectors for a O₂, CO₂ and other monitors.

In another preferred embodiment, the access ports can include gloves inductively welded to removable connection fittings which are fixed to the access ports.

In use, the isolette is removed from a storage bag. The flexible enclosure is fitted to the stanchions and secured to the perimeter of the base panel. The patient is positioned in the isolette enclosure. The drape is positioned over the patient. The drape is fixed in position around the patient and held in position by a strap, tape or by tucking the drape sides under the patient. The pressurization port is connected to a vacuum turbine. Negative pressure is applied to the pressurization port by the turbine, thereby evacuating the isolette. The patient may be accessed through integrated gloves sealed to the access ports or by resealable flaps covering the access ports. Leads and tubes can be inserted into the isolette through the component access panel(s) which can be located on either side or the front of the isolette.

In other preferred embodiments, the isolette comprises an internal support frame having identical right and left sides. The left and right sides are each removably fixed to side rails of a patient bed. The support frame suspends an integrated top subassembly. The top subassembly is sealed to a drape subassembly. The top subassembly includes two opposing side walls, a front panel and a top panel. The side panels include pockets which removably accommodate the support frames. A pressurization port is provided in one or both of the side walls. Resealable access panels are provided in the side and front panels. A component access panel is provided. The drape subassembly may include a seal ring and a securing strap to be secured to a patient. The access ports may include integrated or removable bi-directional gloves.

In use, the support frames are attached to opposing bed rails. The top subassembly is fixed to the support frames. The patient is positioned inside the isolette. The drape is extended over the patient. The sealing ring is positioned around the patient and secured by the strap. Negative pressure is then supplied by a turbine connected to the isolette through the pressurization port.

### BRIEF DESCRIPTION OF THE DRAWINGS

In the detailed description of the preferred embodiments presented below, reference is made to the accompanying drawings.

FIG. 1 is an isometric drawing of a preferred embodiment of an isolette.

FIG. 2 shows an exploded isometric view of a drape subassembly

FIG. 3 shows an exploded isometric view of a base subassembly.

FIG. 4A is detail view of an alternate preferred embodiment of a side panel.

FIG. 4B is detail view of an alternate preferred embodiment of a side panel.

FIG. 5A is an exploded isometric view of a preferred component access panel.

FIG. 5B is an exploded isometric view of an alternate embodiment of an access panel.

FIG. 6 is an isometric view of an alternate embodiment of a component access panel.

FIG. 7A is an exploded isometric view of a preferred glove connection.

FIG. 7B is an isometric view of a preferred glove connection.

FIG. 7C is an isometric view of a preferred glove connection.

FIG. 7D is an exploded isometric view of a preferred glove connection.

FIG. 7E is an exploded isometric view of a preferred barrier cap.

FIG. 8 is a side view of a preferred embodiment of an isolette is use.

FIG. 9 is an isometric view of a preferred embodiment of an alternate embodiment.

FIG. 10A is an exploded isometric view of a preferred embodiment of a top subassembly.

FIG. 10B is an isometric view of a detail of the top subassembly.

FIG. 11 is an exploded isometric view of a preferred embodiment of a frame subassembly.

FIG. 12A is an isometric view of a preferred embodiment of a side bracket.

FIG. 12B is an isometric view of a preferred embodiment of a side bracket.

FIG. 13 is an isometric view of an alternate embodiment of an isolette.

FIG. 14 is an exploded isometric view of an alternate embodiment of a top subassembly.

FIG. 15 is an isometric view of an alternate embodiment of a frame assembly.

FIG. 16A is a flowchart of a preferred method of use of a preferred embodiment of an isolette.

FIG. 16B is a flowchart of a preferred method of use of a preferred embodiment of an isolette assembly.

FIG. 16C is a flowchart of a preferred method of use of a preferred embodiment of an isolette assembly.

FIG. 16D is a flowchart of a preferred method of use of a preferred embodiment of an isolette assembly.

### DETAILED DESCRIPTION OF THE INVENTION

In the description that follows, like parts are marked throughout the specification and figures with the same numerals, respectively. The figures are not necessarily drawn to scale and may be shown in exaggerated or generalized form in the interest of clarity and conciseness.

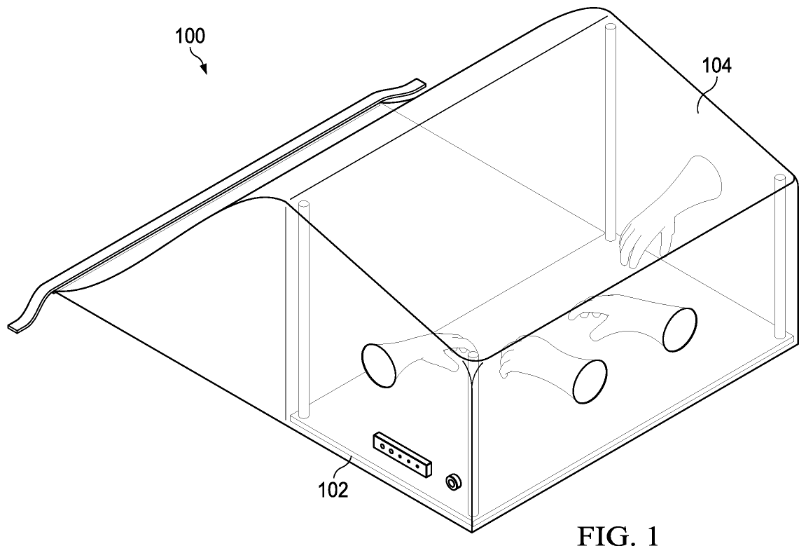

Referring to FIG. 1, isolette 100 is further described. Isolette 100 is comprised of base subassembly 102 and top subassembly 104.

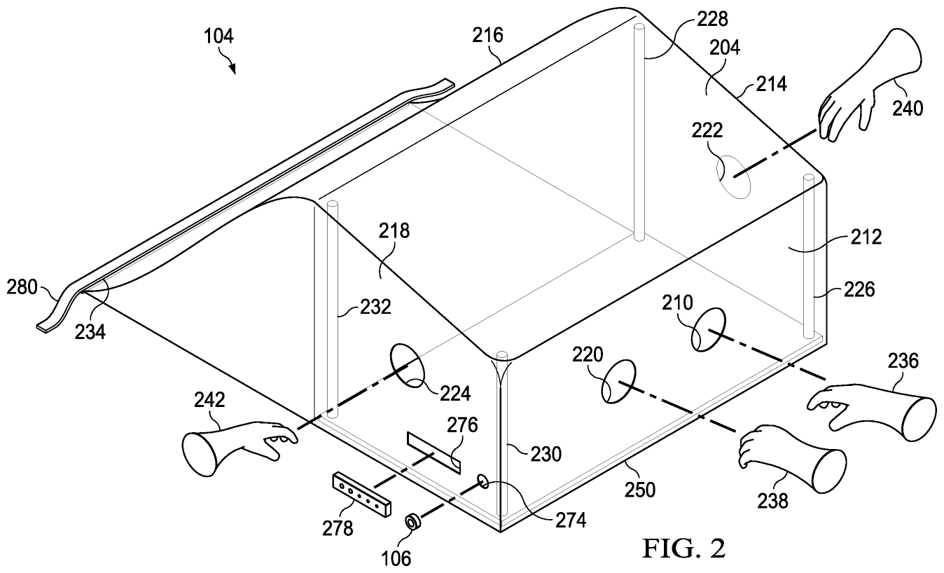

Referring to FIG. 2, top subassembly 104 includes side panel 214, side panel 218, front panel 212, bottom panel 250, top panel 204, and drape 216. Front panel 212 is connected to side panel 214 and side panel 218. Top panel 204 is connected to side panels 214 and 218, front panel 212, and drape 216. Bottom panel 250 is connected to side panel 218, front panel 212 and side panel 214. Front stanchion pocket 226 is formed in side panel 214 adjacent front panel 212. Rear stanchion pocket 228 is formed in side panel 214 adjacent drape 216. Front stanchion pocket 230 is formed in side panel 218 adjacent front panel 212. Rear stanchion pocket 232 is formed in side panel 218 adjacent drape 216. Each of the stanchion pockets is flexible and generally vertical and about ⅜ inch to 1 inch in diameter.

Side panel 218 further includes access port 224, hole 276, and hole 274. Bi-directional glove 242 is attached to the outside face of side panel 218, within access port 224. Bi-directional glove 242 may be used with either hand and is positioned with the thumb directed upward with respect to the isolette. Component access panel 278, which will be further described, is adhered to side panel 218 at hole 276 by medical grade adhesive. Pressure fitting 106 is adhered to the outside face of side panel 218, adjacent hole 274. Pressure fitting 106 is preferably a slip tip connector, tapered nipple or other form of quick disconnect coupling. The component access panel and pressure fittings may be positioned in any or all of the panels.

Front panel 212 includes access ports 220 and 210. Bi-directional glove 238 is adhered to the outside face of front panel 212 within access port 220. Bi-directional glove 236 is adhered to the outside face of front panel 212 within access port 210. Side panel 214 further includes access port 222. Bi-directional glove 240 is adhered to the outside face of side panel 214, within access port 222. Each of the gloves is adhered at the glove sleeve.

Drape 216 is attached to top panel 204 and side panels 214 and 218. In general, the drape forms a 3-sided enclosure adapted to cover the upper quarter of the patient. Drape 216 may include an optional flexible seal 234 adhered adjacent its distal edge. Flexible seal 234 is preferably constructed from closed cell butyl foam rubber. Strap 280 is adhered to the length of flexible seal 234. Strap 280 is preferably constructed from a woven fabric of length sufficient to encircle the front torso of the patient. Preferably the strap is secured by a Velcro® type closure or tension buckles at opposite ends and is adapted to compress the flexible seal around the patient and secure it in place.

In a preferred embodiment, side panels 214 and 218, bottom panel 250, front panel 212, top panel 204, and drape 216 are constructed of transparent or semitransparent polyethylene sheeting between 8 and 30 mils thick. In a preferred embodiment, the panels and drape may be integrally formed in a bag-like structure with a rear opening for the patient. The internally formed version of the invention is preferable because it assists in containing contagions. In another preferred embodiment, the panels and the drape may be connected by inductive welding at the seams.

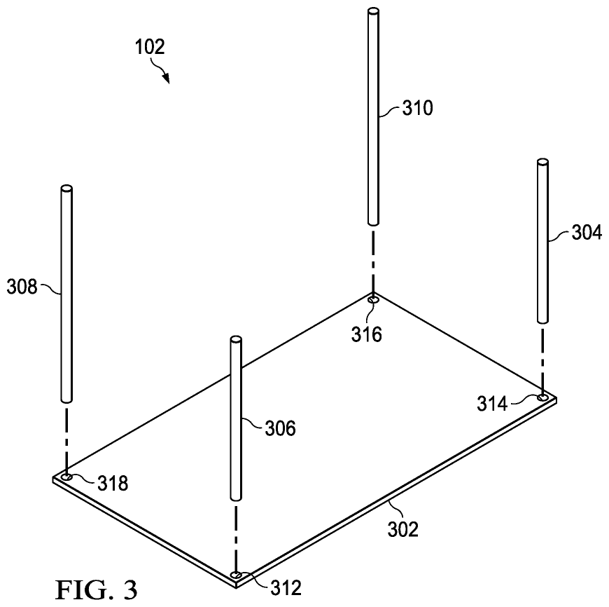

Referring to FIG. 3, base subassembly 102 will be further described. Base panel 302 is generally a flat rectangular sheet of light, disposable corrugated material. Base panel 302 is preferably constructed of semi-rigid corrugated thermoplastic sheet, preferably of corriboard or coroplast sheeting approximately ⅛ inch thick.

Base subassembly 102 includes base panel 302, front stanchion 306, front stanchion 304, front stanchion 306, rear stanchion 308 and rear stanchion 310.

Front stanchion 306 is attached to base panel 302 at joint 312. Front stanchion 304 is attached to base panel 302 at joint 314. Rear stanchion 310 is attached to base panel 302 at joint 316. Rear stanchion 308 is attached to base panel 302 at joint 318. Front stanchions 304 and 306 are preferably about 16 inches in height. Rear stanchions 308 and 310 are about 18 inches in height. The differing heights of the front and rear stanchions is important because they impart a downward sloping angle to the top panel toward the front panel, which aids in viewing the patient from the front of the isolette. Rear stanchions 308 and 310 and front stanchions 304 and 306 are preferably constructed of fiberglass, aluminum or stainless steel rod stock about ⅜ inches in diameter.

Joints 312, 314, 316, and 318 preferably include a medical grade adhesive or a threaded connection.

When assembled, front stanchion pockets 226 and 230 are fitted over front stanchions 304 and 306, respectively. Rear stanchion pockets 228 and 232 are fitted over rear stanchions 310 and 308, respectively. Bottom panel 250 rests on base panel 302 and may be secured by adhesive strips. The stanchions support the panels and the drape thereby forming the isolette. In another embodiment, side panels 214 and 218, and front panel 212 are also secured to the perimeter of base panel 302. In a preferred embodiment, side panels 218 and 214 and front panel 212 are secured to base panel 302 with a suitable medical adhesive.

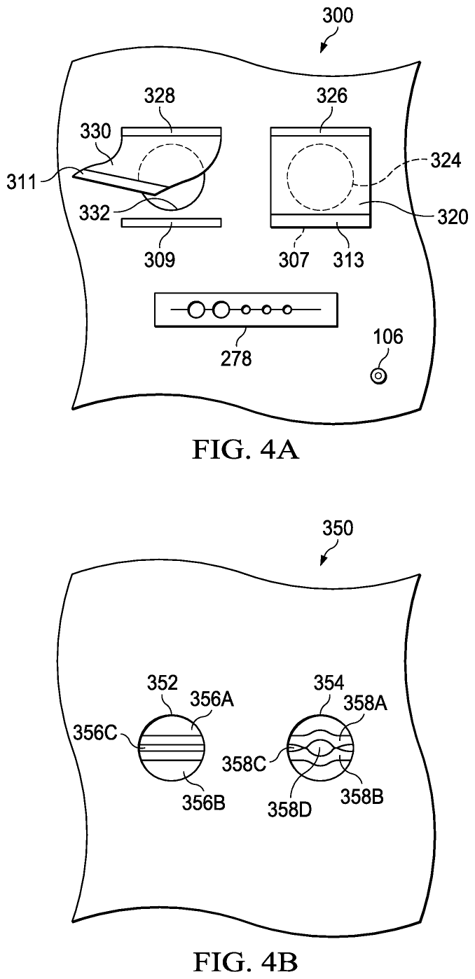

Referring to FIG. 4A, an alternate embodiment of a panel, will be further described. All versions of the panels disclosed may optionally be employed in all the various embodiments of the isolette in either side, top or front panel positions.

Panel 300 further comprises access ports 332 and 324. In this example, two (2) access ports, a single component access panel 278 and a single pressure fitting 106 are provided in the panel. However, a single access port in the panel may be provided. In yet other embodiments, no access ports are provided in the panel, but are created as needed on an ad hoc basis, as will be further described. Flap 330 is fixed to panel 300 adjacent to and covering access port 332. Flap 330 is fixed to the panel by adhesive 328. Flap 330 further comprises closure 311. Panel 300 further include closure 309. In use, closure 311 is removably affixed to closure 309, thereby sealing flap 330 over access port 332.

Likewise, flap 320 is positioned adjacent to and covering access port 324. Flap 320 is fixed to panel 300 via adhesive 326. Closure 307 is fixed to panel 300. Closure 313 is fixed to flap 320. In use, closure 313 is adapted to removably seal against closure 307, thereby sealing flap 320 over access port 324.

The closures preferably include Velcro® type closures, Ziplock® type closures or resealable adhesive straps.

Referring then to FIG. 4B, an alternate embodiment of a panel will be further described.

Panel 350 is further comprised of access ports 352 and 354. Access port 352 further comprises iris diaphragms 356a and 356b. Iris diaphragm 356a overlaps iris diaphragm 356b, in region 356c.

Access port 354 further comprised of iris diaphragm 358a and 358b. Iris diaphragm 358a overlaps iris diaphragm 358b, in region 358c. Iris diaphragms 358a and 358b, are shown in an open position, accommodating opening 358d.

In a preferred embodiment, the iris diaphragms are comprised of butyl rubber sheet or low-density polyethylene (LDPE), approximately 10 mils in thickness. The iris diaphragms are fixed to the exterior of the access port, except for the overlap region, which may be parted by physical pressure, to create a ducted portal in the access port, such as opening 358d.

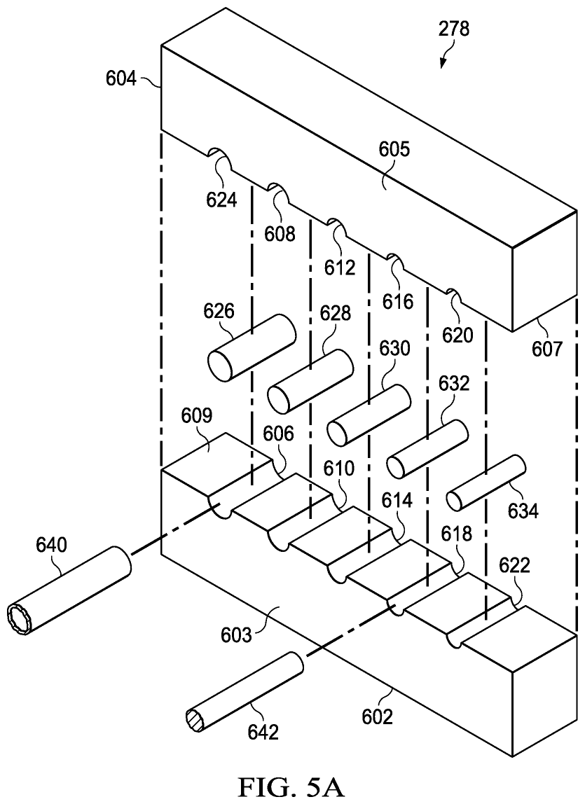

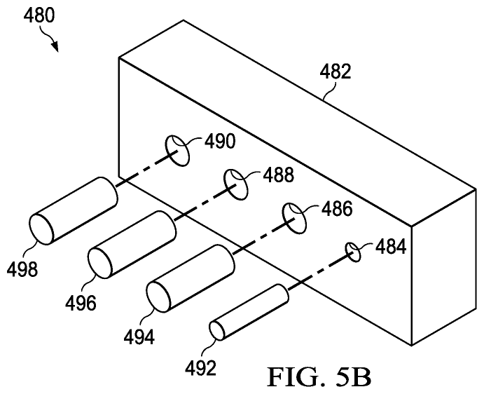

Referring to FIG. 5A, a preferred embodiment of the component access panel will be further described. Component access panel 278 includes top section 604 and bottom section 602. Top section 604 further comprises front face 605 and bottom face 607. Bottom section 602 further comprises front face 603 and top face 609. When assembled, front face 605 and front face 603 are positioned adjacent the inside surface of the side panel and affixed with a suitable medical adhesive. Bottom face 607 is positioned adjacent top face 609.

Semi-cylindrical slots 606, 610, 614, 618, and 622 are formed in the bottom section. Semi-cylindrical slots 624, 608, 612, 616, and 620 are formed in the top section adjacent semi-cylindrical slots 606, 610, 614, 618, and 622, respectively. In general, the slots are all generally parallel and all generally perpendicular to the side panel. When the top and bottom sections are assembled, resealable plug 626 fits within slots 606 and 624. Resealable plug 628 fits within slots 608 and 610. Resealable plug 630 fits within slots 612 and 614. Resealable plug 632 fits within slots 616 and 618. Resealable plug 634 fits within slots 620 and 622. The plugs prevent gas leakage through the access panel. In a preferred embodiment, the slots are adapted to seal against the outside surface of different sized tubes and wires, thereby preventing air/gas leakage through the access panel by insertion of tubes 640 and wires 642, as will be further described. In a preferred embodiment, component access panel 278 is approximately ¼ inch×1 inch cross-section and approximately 8 inches in length and is comprised of closed cell neoprene rubber foam.

Referring to FIG. 5B, an alternate embodiment of the component access panel will be described.

Access panel 480 is comprised of a single integrated block 482. Integrated block 482 includes through holes 484, 486, 488 and 490. Removable plugs 492, 494, 496 and 498 are positioned in through holes 484, 486, 488 and 490. Preferably, the removable plugs are integrally formed with block 482 and held in place by perforations. The block is preferably comprised of closed cell neoprene rubber foam.

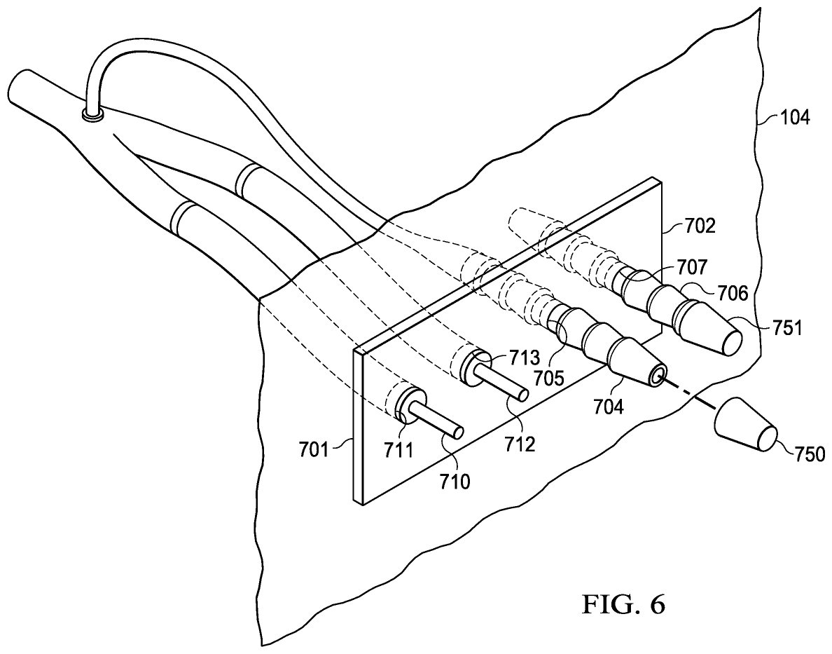

Referring to FIG. 6, an alternate embodiment of the component access panel will be described. Component access panel 702 is generally a semi-rigid plastic connector block supporting several hose connectors. Component access panel 702 further comprises rear surface 701, adhered to side panel 104 with a suitable adhesive.

Component access panel 702 preferably includes a number of double ended connectors forming ducted portals from the exterior of the isolette to its interior. For example, component access panel 702, in this embodiment, includes circuit connector 710, circuit connector 712, circuit connector 704, and circuit connector 706. Circuit connector 710 and circuit connector 712 are adapted to connect to anesthesia lines. Circuit connector 704 and circuit connector 706 are adapted to connect to a source of vacuum. Circuit connector 710 passes through component access panel 702 thru through hole 711. Likewise, circuit connectors 712, 704 and 706 pass through component access panel 702 via through holes 713, 705 and 707, respectively.

In this example, the circuit connectors are adapted to be sealed by caps when not in use, such as cap 750 adapted to mate with circuit connector 704, and cap 751 shown in place on circuit connector 706. In this example, the circuit connectors are either wedge type, slip tip, barb type or Luer lock type connectors. Other connector types may be used equally well.

In a preferred embodiment, the component access panel is comprised of a semi flexible polyvinyl chloride sheet, approximately 100 mils thick. The circuit connectors may be adhered in the through, or micro-perforated, holes with a suitable medical grade adhesive sealant.

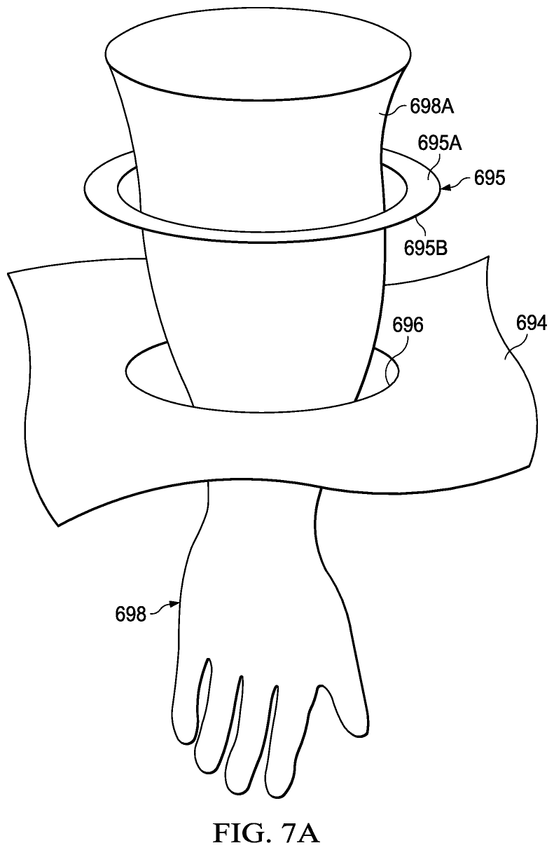

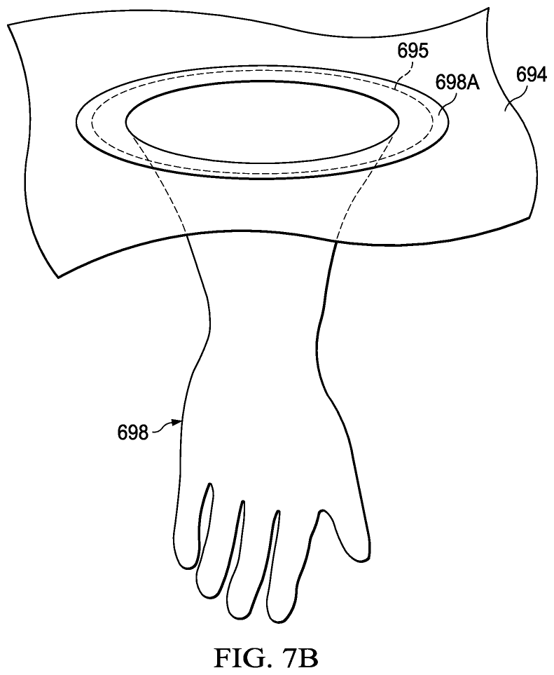

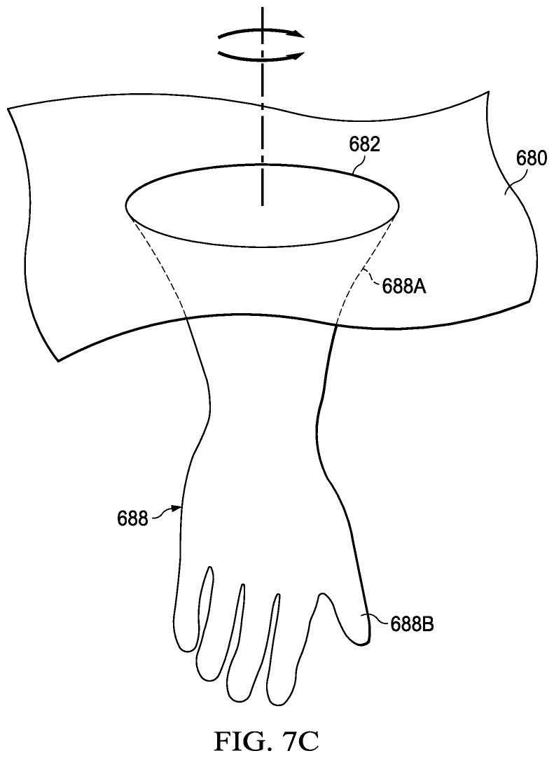

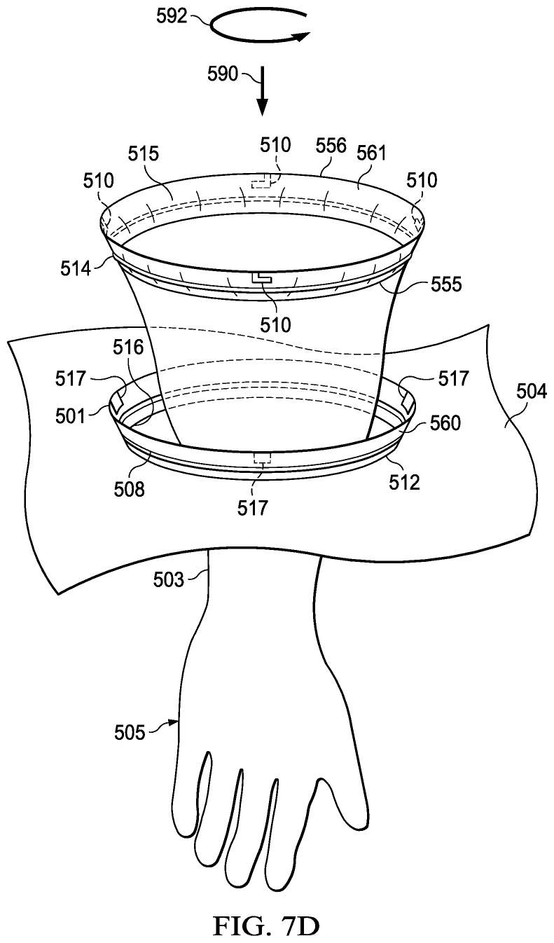

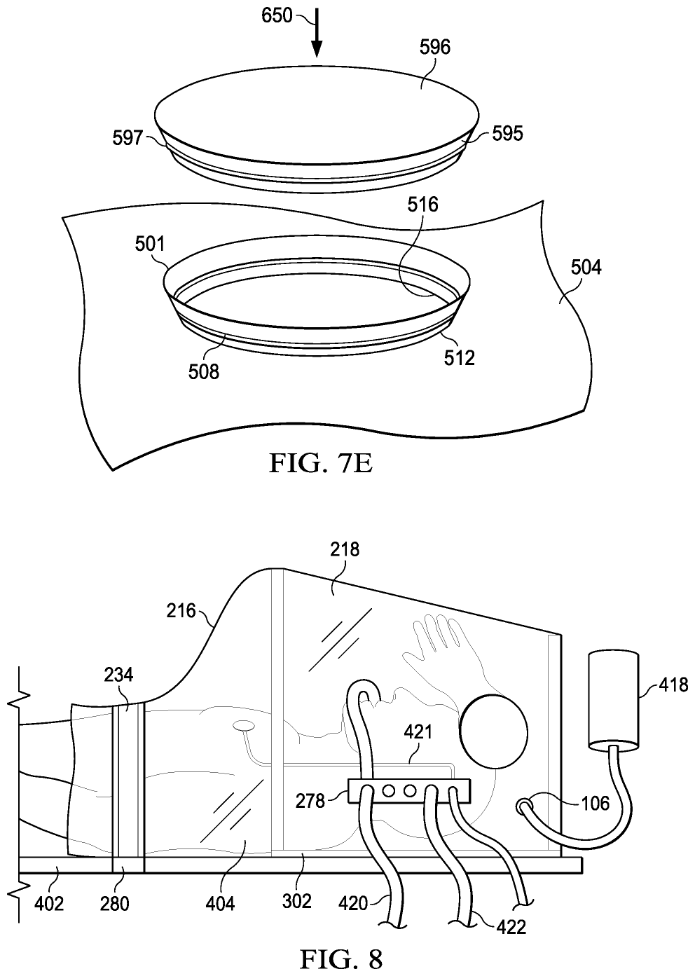

Referring to FIGS. 7A and 7B, an alternate embodiment of an access port will be described. Each embodiment of an access port described may be used with all of the access panels of the various embodiments of the isolette.

Panel 694 includes access port 696. Adhesive ring 695, is adapted to circumscribe access port 696. Adhesive ring 695 further comprises adhesive surface 695a and adhesive surface 695b. Glove 698 is adapted to fit within access port 696 and within adhesive ring 695.

Referring then to FIGS. 7A and 7B, when assembled, glove sleeve 698a is fixed to adhesive surface 695a. Panel 694 is fixed to adhesive surface 695b. The connections between glove sleeve 698a, adhesive surface 695a, adhesive surface 695b and side panel 694, create an airtight seal, thereby allowing access to the glove from the exterior of the isolette without release of contagions within the isolette. This embodiment is important, because the glove can be placed in any location, in a side, or top panel, on an ad-hoc basis, as will be further described.

Referring thing to FIG. 7C, an alternate embodiment of an access port will be described. Panel 680 includes access port 682. Panel 680 is fused and sealed to glove sleeve 688a of glove 688, around access port 682, thereby allowing access to the glove from the exterior of the isolette. In this embodiment, glove thumb 688b is oriented vertically upward with respect to the isolette, when fused to access port 682. Preferably, glove 688 is a bi-directional glove which may be used with either the left, or the right, hand. The upward orientation of the glove is important, because it allows the care giver to rapidly insert either hand into the glove for use without regard to the right handed or left handed nature of the glove.

Referring to FIG. 7D, an alternate embodiment of an access port will be described. Panel 504 includes connection ring 512 sealed adjacent access port 516 along annular interface 501. The connection ring is sealed at the annular interface with suitable medical adhesive. Connection ring 512 is further comprised of frustroconical body 560. Frustroconical body 560 includes annular O-ring channel 508 on its interior surface. Frustroconical body 560 further includes a plurality of bayonet mount pins 517, dispersed at 90° angles on the interior surface of the frustroconical body relative to a central axis of the frustroconical body.

Glove collar 556 is designed to mate with connection ring 512. Glove collar 556 includes frustroconical body 561. Frustroconical body 561 further includes annular O-ring 514 compressed into exterior annular channel 515. The frustroconical body further includes bayonet mount receptors 510.

Glove 505 is attached to glove collar 556 at sleeve 503 and annular interface 555. In preferred embodiments, sleeve 503 is adhered to annual interface 555 with a suitable medical grade adhesive.

In use, glove 505 is advanced through connection ring 512 and into the interior of the isolette. Glove collar 556 advances in direction 590 into connection ring 512 where annular O-ring 514 seats in O-ring channel 508, thereby forming an air-tight seal. Once the O-ring is seated, glove collar 556 is rotated in direction 592 to lock bayonet mount receptors 510 into position adjacent bayonet mount pins 517.

Referring to FIG. 7E, an alternate embodiment of a connection ring, will be further described. Connection ring 512 includes O-ring channel 508 adjacent its interior annular surface. Barrier cap 596 includes frustroconical body 595 and is generally adapted to seal to the connection ring. Frustroconical body 595 includes annular O-ring 597. O-ring 597 is adapted to seat within O-ring channel 508 as barrier cap 596 is advanced into access port 516 in direction 650. Barrier cap 596 is removably fixed in the access point and creates an airtight seal between the exterior of the isolette and the interior of the isolette.

Referring to FIG. 8, in use, patient 404 is positioned on flat surface 402, such as a gurney or hospital bed. The patient's head and shoulders rest on bottom panel 250 and base panel 302 between side panel 218 and side panel 214. Drape 216 is extended over the patient. Flexible seal 234 is positioned around the patient. Strap 280 is secured around the seal. Pressure fitting 106 is attached to turbine 418. The turbine supplies negative pressure to the isolette. In an extremely rare case, the turbine may supply a positive pressure to the isolette. In use, isolette 100 is capable of supporting negative pressure in the range about −0.1 to −0.01 mmHg or approximately 2 kPa. Isolette 100 is capable of supporting positive pressure of about 0.01 to about 0.1 mmHg or about 0.2 kPa.

Oxygen line 420 passes through component access panel 278 and is secured in place by a friction fit when O₂ is used. Suction line 422 passes through component access panel 278 where it also is secured with a friction fit. Likewise, ERG lead 421 passes through component access panel 278. Of course, the component access panel may accommodate other patient support tubes and leads as required.

The patient may be accessed through any of the access ports by use of the integrated gloves or cover flaps.

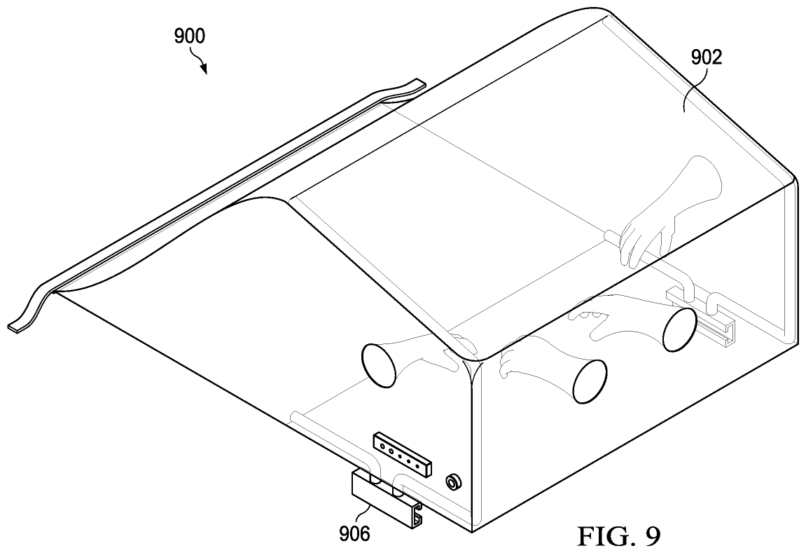

Referring to FIG. 9, an alternate embodiment of isolette 900 will be further described. Isolette 900 includes frame subassembly 906 and top subassembly 902.

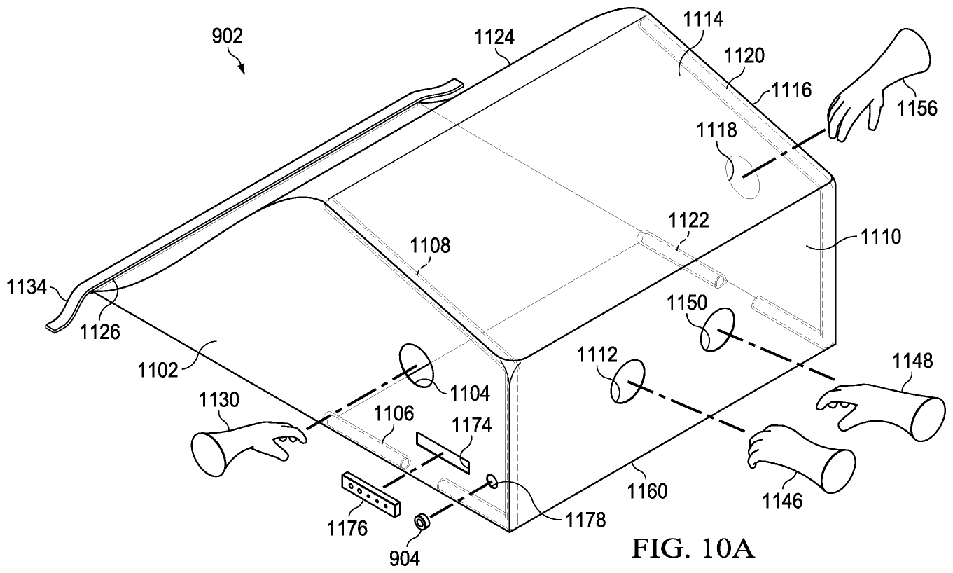

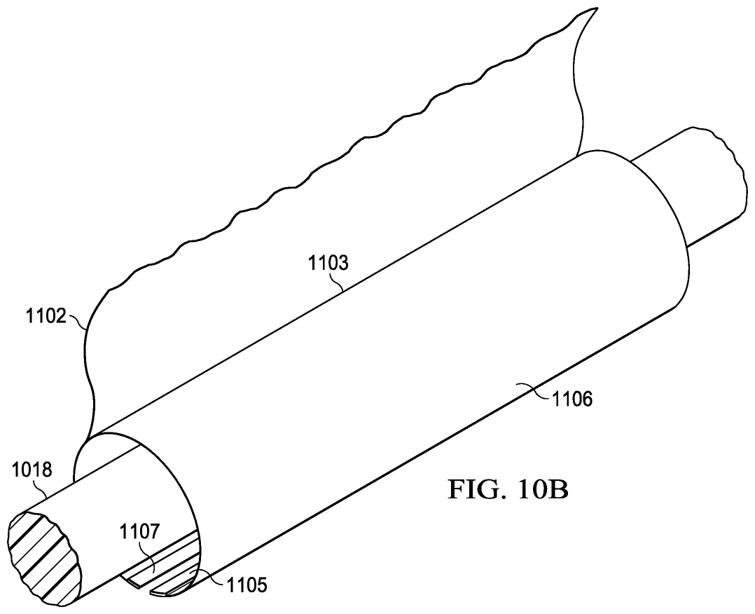

Referring to FIG. 10A, top subassembly 902 will be further described. Top subassembly 902 includes side panel 1102, side panel 1116, front panel 1110, bottom panel 1160, top panel 1114 and drape 1124.

Front panel 1110 is attached to bottom panel 1160, side panel 1102, top panel 1114, and side panel 1116. Front panel 1110 includes access port 1112 and access port 1150. The access ports each are holes in the plastic sheeting. Bi-directional glove 1146 is adhered, in a thumb-up orientation, to front panel 1110 within access port 1112. Bi-directional glove 1148 is adhered, in a thumb-up orientation, to front panel 1110 within access port 1150.

Side panel 1102 includes access port 1104, hole 1174, hole 1178, rear support pocket 1106, and front support pocket 1108. Rear support pocket 1106 is a tubular structure sized to accommodate the rear horizontal support, as will be further described. Front support pocket 1108 is a tubular structure positioned around the perimeter of side panel 1102 and sized to accommodate the front horizontal support, the front vertical support and the front angled support, as will be further described. Bi-directional glove 1130 is adhered in a thumb-up orientation to side panel 1102 within access port 1104. Component access panel 1176, is adhered to side panel 1102 covering hole 1174, as will be further described. Connector 904 is adhered to side panel 1102 covering hole 1178.

Side panel 1116 includes access port 1118, rear support pocket 1122, and front support pocket 1120. Rear support pocket 1122 is a tubular structure sized to accommodate the rear horizontal support, as will be further described. Front support pocket 1120 is a tubular structure positioned around the perimeter of side panel 1116 and sized to accommodate the front horizontal support, the front vertical support, and the front angle support, as will be further described. Bi-directional glove 1156 is adhered, in a thumb-up orientation, to side panel 1116 adjacent access port 1118.

Base panel 1160 is attached to side panel 1102, front panel 1110 and side panel 1116.

Drape 1124 is attached to top panel 1114 and side panels 1102 and 1116. When seal strip 1126 is used, it is distally adhered to the underside of drape 1124. Seal strip 1126 is constructed of a flexible closed cell butyl rubber. Strap 1134 is adhered to the upper side of drape 1124 adjacent seal strip 1126. Strap 1134 is of length sufficient to surround the patient's torso and is preferably constructed from a woven material with a Velcro® type closure at each end.

In a preferred embodiment, side panels 1102 and 1116, front panel 1110, bottom panel 1160, top panel 1114, and drape 1124 are constructed of transparent or semitransparent polyethylene sheeting between 4 and 30 mils thick. In a preferred embodiment, the panels and drape may be integrally formed. In another preferred embodiment, the panels and the drape may be attached by inductive welding.

Referring to FIG. 10B, rear support pocket 1106 will be further described. Rear support pocket 1106 is a generally cylindrical structure, with an open side, adapted to encircle rear horizontal support 1018. Rear support pocket 1106 further includes parallel closures 1105 and 1107 along the open side. Closures 1105 and 1107 are preferably Velcro® type closures but may also be easily a releasable medical adhesive.

Side panel 1102 is fixed to rear support pocket 1106 at joint 1103. Preferably joint 1103 is accomplished through inductive welding.

Rear support pocket 1122 is of similar structure and is adapted to removably encircle rear horizontal support 1038, as will be further described.

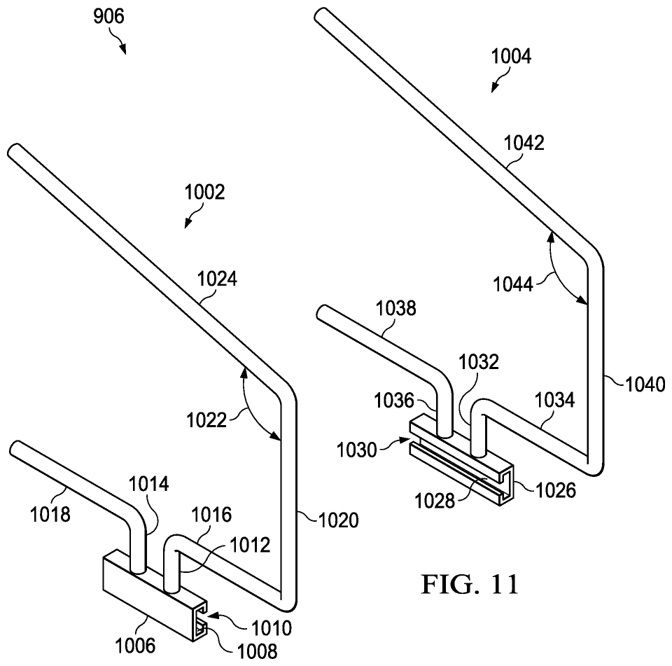

Referring to FIG. 11, frame subassembly 906 will be further described. Frame subassembly 906 includes left support frame 1002 and right support frame 1004.

Left support frame 1002 includes rear horizontal support 1018, rear support stanchion 1014, bracket 1006, front support stanchion 1012, front horizontal support 1016, front vertical support 1020 and front angle support 1024. Bracket 1006 is preferably constructed of aluminum channel 1008 having longitudinal gap 1010.

Bracket 1006 is rigidly attached to rear support stanchion 1014 at about a right angle. Rear support stanchion 1014 is preferably integrally formed with rear horizontal support 1018. Bracket 1006 is also rigidly attached to front support stanchion 1012 at about a right angle. Front support stanchion 1012 is preferably integrally formed with front horizontal support 1016 at about a right angle. Front horizontal support 1016 is preferably integrally formed with front vertical support 1020 at about a right angle. Front vertical support 1020 is preferably integrally formed with front angle support 1024 at angle 1022. Angle 1022 is between approximately 90° and approximately 135°.

Right support frame 1004 comprises rear horizontal support 1038, rear support stanchion 1036, bracket 1026, front support stanchion 1032, front horizontal support 1034, front vertical support 1040 and front angle support 1042. Bracket 1026 is preferably constructed of channel 1028 having longitudinal gap 1030. Bracket 1026 is rigidly attached to rear support stanchion 1036 and front support stanchion 1032 at preferably right angles. Rear support stanchion 1036 is integrally formed with rear horizontal support 1038 at about a right angle. Front support stanchion 1032 is preferably integrally formed with front horizontal support 1034 at about a right angle. Front horizontal support 1034 is preferably integrally formed with front vertical support 1040 at about a right angle. Front vertical support 1040 is preferably integrally formed with front angle support 1042 at angle 1044. Angle 1044 is between approximately 90° and approximately 135° and approximately the same as angle 1022.

All of the supports and stanchions of left support frame 1002 are generally coplanar. Likewise, all of the supports and stanchions of right support frame 1004 are generally coplanar. In a preferred embodiment, right support frame 1004 is a mirror image of left support frame 1002. Right support frame 1004 and left support frame 1002 are preferably between 18 inches and 24 inches in height. Angles 1022 and 1044 are important because they impart a downward sloping orientation of the top panel toward the front of the isolette, which aids in easily seeing the patient from the front when the isolette is in use.

In a preferred embodiment, right support frame 1004 and left support frame 1002 are constructed from aluminum, fiberglass and/or stainless steel rod stock about ⅜ inch in diameter.

In a preferred embodiment, bracket 1006 and bracket 1026 are formed from 2 inch aluminum or stainless steel C-channel stock.

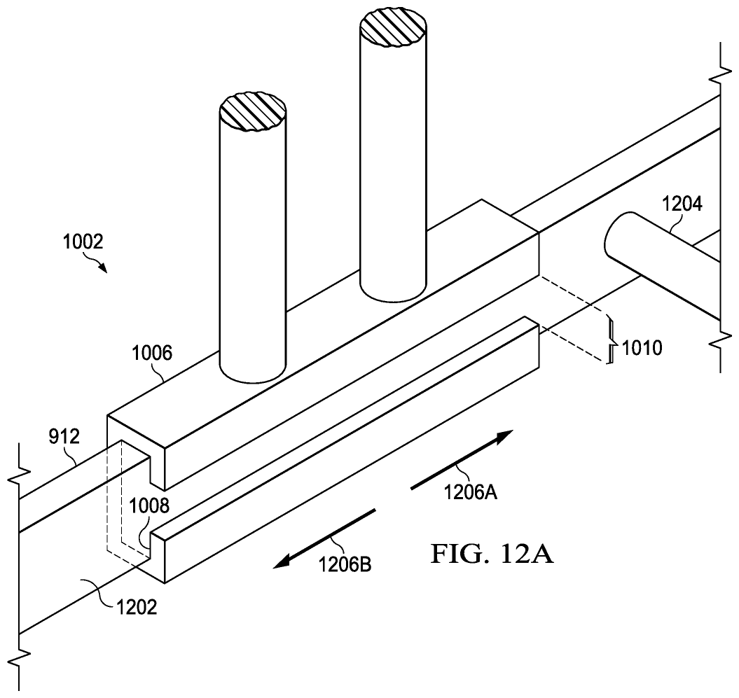

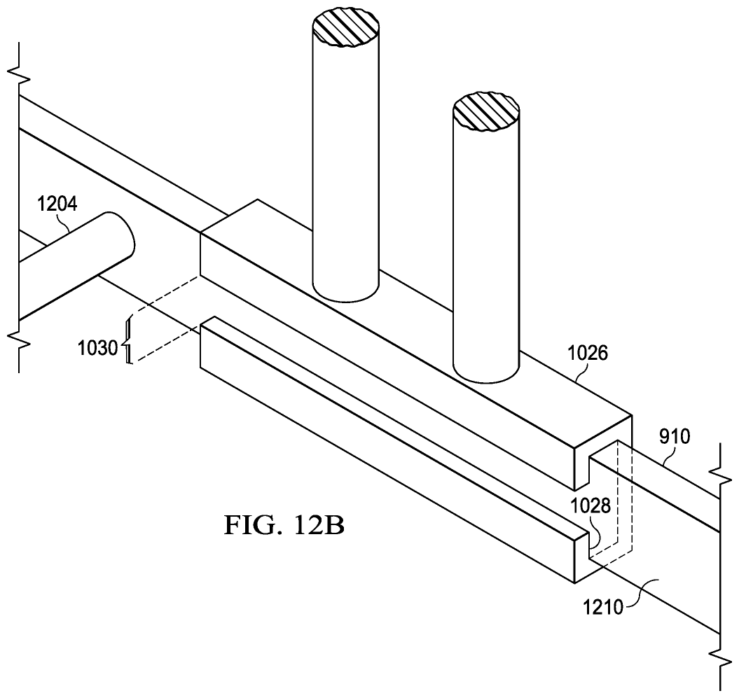

Referring to FIGS. 12A and 12B, brackets 1006 and 1026 will be further described. Side rails 912 and 910 include interior rail surfaces 1202 and 1210, respectively and bed rail connector 1204. Channel 1008 is positioned adjacent side rail 912. Channel 1028 is positioned adjacent side rail 910. Gap 1010 is preferably positioned adjacent interior rail surface 1202. Gap 1030 is preferably positioned adjacent interior rail surface 1210. Brackets 1006 and 1026 may be adjusted along side rails 912 and 910 respectively, in either of directions 1206a or 1206b. In a preferred embodiment, the gaps are adapted to accommodate the bed rail connectors so that the brackets move past bed rail connector 1204 as their positions are changed.

To assemble isolette 900, front angle support 1024, front vertical support 1020, and front horizontal support 1016 are positioned in front support pocket 1108. Front angle support 1042, front vertical support 1040, and front horizontal support 1034 are positioned in front support pocket 1120. Rear support pocket 1106 is closed around rear horizontal support 1018. Rear support pocket 1122 is closed around rear horizontal support 1038. Bracket 1006 is positioned alongside side rail 912, and bracket 1026 is positioned along side rail 910, opposite bracket 1006, the brackets are then secured in place, on the bed rails, preferably with a thumb screw.

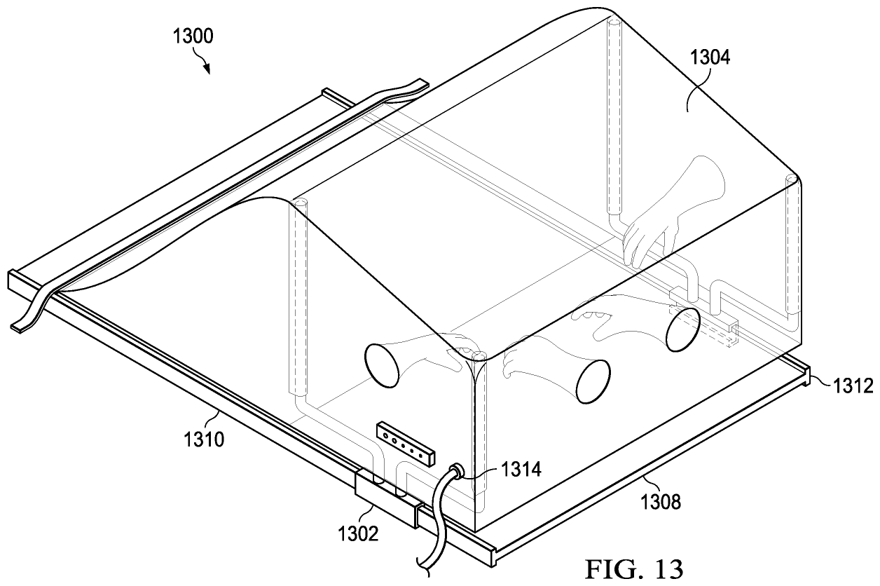

Referring to FIG. 13, an alternate embodiment isolette 1300 will be further described. Isolette 1300 includes frame subassembly 1302 and top subassembly 1304. Isolette 1300 is positioned on table 1308. Table 1308 includes side rail 1310 and side rail 1312.

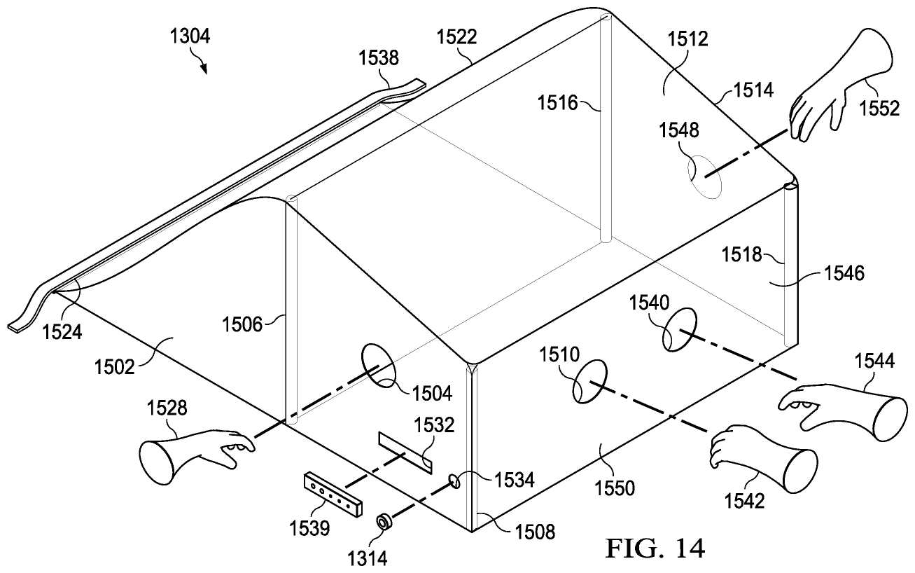

Referring to FIG. 14, top subassembly 1304 will be further described. Top subassembly 1304 includes side panel 1502, front panel 1546, side panel 1514, base panel 1550, top panel 1512 and drape 1522.

Front panel 1546 is connected to side panel 1502, side panel 1514, bottom panel 1550, and top panel 1512. Front panel 1546 includes access port 1510 and access port 1540. Bi-directional glove 1542 is adhered to front panel 1546 inside access port 1510 in a thumb-up orientation. Bi-directional glove 1544 is adhered to front panel 1546 inside access port 1540 in a thumb-up orientation,

Side panel 1502 is connected to bottom panel 1550, top panel 1512 and front panel 1546. Side panel 1502 includes rear support pocket 1506, front support pocket 1508, access port 1504, hole 1532, and hole 1534. The support pockets each are generally vertical flexible tubular structures integrally formed with or inductively welded to the interior of the side panel. Rear support pocket 1506 is sized to accommodate rear vertical support 1422. Front support pocket 1508 is attached to side panel 1502 adjacent front panel 1546. Front support pocket 1508 is sized to accommodate front vertical support 1416. Bi-directional glove 1528 is attached to side panel 1502 within access port 1504 in a thumb-up orientation. Component access panel 1539 is adhered to side panel 1502 covering hole 1532, as previously described. Pressure port 1314 is attached to side panel 1502 adjacent hole 1534, as previously described. In other embodiments, the component access panel and the pressure port may reside in other side or front panels.

Side panel 1514 is connected to bottom panel 1550, top panel 1512 and front panel 1546. Side panel 1514 includes rear support pocket 1516, front support pocket 1518 and access port 1548. Rear support pocket 1516 is sized to accommodate rear vertical support 1440. Front support pocket 1518 is attached to side panel 1514 adjacent front panel 1546 and is sized to accommodate front vertical support 1434. Bi-directional glove 1552 is attached to side panel 1514 within access port 1548 in a thumb-up orientation.

Drape 1522 is attached to top panel 1512, and side panels 1502 and 1514. Optional flexible seal 1524 is fixed laterally at the distal end of drape 1522. Flexible seal 1524 is preferably constructed of closed cell butyl rubber. Strap 1538 is located adjacent flexible seal 1524. In a preferred embodiment, strap 1538 is of sufficient length to surround the patient and compress the flexible seal around the patient. The strap is preferably constructed of a woven material and includes Velcro® type closures or adhesive closures. In another embodiment, the drape may be secured with medical adhesive tape. In another embodiment, the drape may be secured to the patient by simply tucking it in.

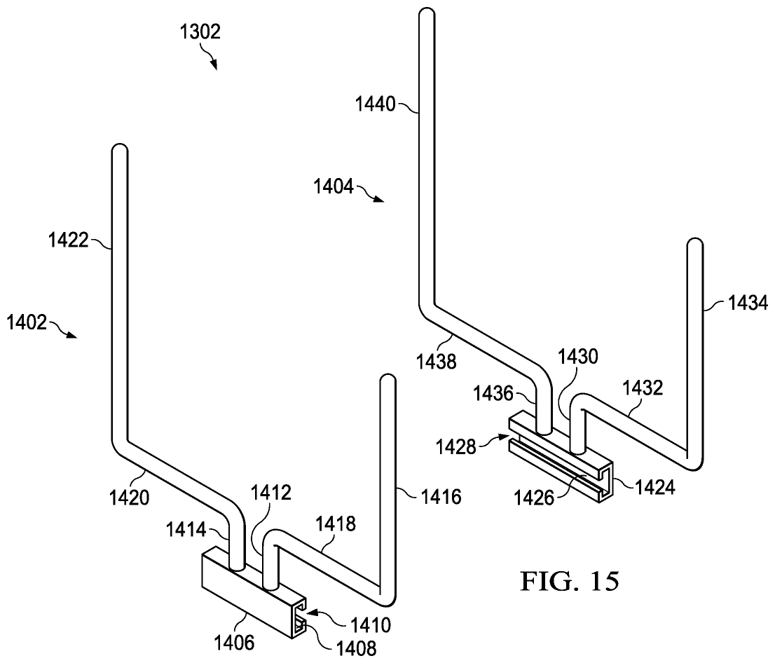

Referring to FIG. 15, frame subassembly 1302 will be further described. Frame subassembly 1302 includes left support frame 1402 and right support frame 1404.

Left support frame 1402 includes bracket 1406. Bracket 1406 includes channel 1408 and gap 1410. Bracket 1406 is attached to rear support stanchion 1414 and front support stanchion 1412. Rear support stanchion 1414 is further attached to rear horizontal support 1420. Rear horizontal support 1420 is further attached to rear vertical support 1422. In a preferred embodiment, rear support stanchion 1414 is integrally formed with rear horizontal support 1420, and rear vertical support 1422. Front support stanchion 1412 is further attached to front horizontal support 1418. Front horizontal support 1418 is further attached to front vertical support 1416. In a preferred embodiment, front support stanchion 1412 is integrally formed with front horizontal support 1418 and front vertical support 1416. All of the supports and stanchions are generally coplanar.

Right support frame 1404 includes bracket 1424. Bracket 1424 includes channel 1426 and gap 1428. Bracket 1424 is attached to rear support stanchion 1436 and front support stanchion 1430. Rear support stanchion 1436 is further attached to rear horizontal support 1438. Rear horizontal support 1438 is further attached to rear vertical support 1440. In a preferred embodiment, rear support stanchion 1436 is integrally formed with rear horizontal support 1438 and rear vertical support 1440. Front support stanchion 1430 is further attached to front horizontal support 1432. Front horizontal support 1432 is further attached to front vertical support 1434. In a preferred embodiment, front support stanchion 1430 is integrally formed with front horizontal support 1432 and front vertical support 1434. All of the supports and stanchions are generally coplanar.

All of the stanchions and supports of left support frame 1402 are generally coplanar. All of the stanchions and supports of right support frame 1404 are also generally coplanar. In a preferred embodiment, right support frame 1404 is a mirror image of left support frame 1404. All attachments occur at generally right angles.

In a preferred embodiment, right support frame 1404 and left support frame 1402 are constructed from aluminum, fiberglass and/or stainless steel rod stock of about ⅜ inch in diameter. In a preferred embodiment front vertical supports 1416 and 1434 are preferably about 18 inches high and rear vertical supports 1422 and 1440 are preferably about 24 inches high. In a preferred embodiment, bracket 1406 and bracket 1424 are formed of 2 inch stainless steel aluminum c-channel stock. The height difference between the front vertical supports and the rear vertical supports is important because it imparts a downward sloping angle of the top panel toward the front of the isolette, which aids in viewing the patient from the front of the isolette when the isolette is in use.

To assemble isolette 1300, rear vertical supports 1422 and 1440 are inserted into rear support pockets 1506 and 1516, respectively. Front vertical supports 1416 and 1434 are inserted into front support pockets 1508 and 1518, respectively. Top subassembly 1304 is secured to brackets 1406 and 1424 with a suitable adhesive. Brackets 1406 and 1424 are secured to the bed rails of the table, as previously described.

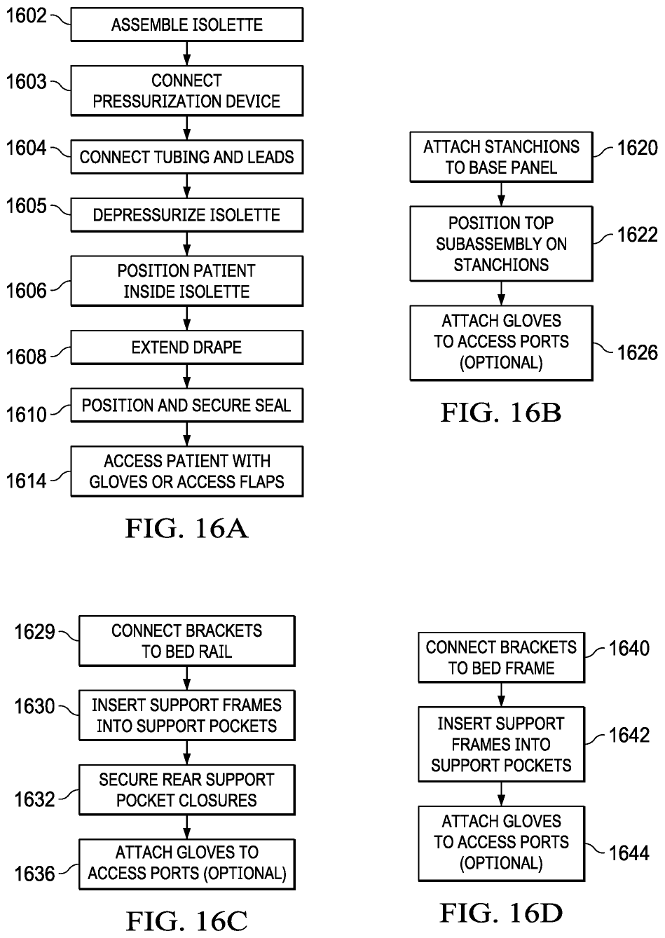

Referring to FIG. 16A, a method of use of isolette 100, will be described.

At step 1602, isolette 100 is assembled, as will be further described.

At step 1603, the turbine is connected to the pressure fitting.

At step 1604, the appropriate sized removable plug is removed from the component access panel by parting the perforations. Leads and tubing are positioned in appropriate access holes. The access panel provides a seal around the wires and tubing to maintain the vacuum inside the isolette.

At step 1605, the turbine is activated and positive or negative pressure is supplied to the isolette.

At step 1606, the patient is positioned on a bed surface in a supine position within the isolette.

At step 1608, the drape is extended over the patient.

At step 1610, the seal is positioned around the patient and secured in place by the strap.

At step 1614, the patient may be accessed through any of the access ports using the integrated flaps, the iris diaphragms, integrated gloves or removable gloves and connection collars and caps.

Referring to FIGS. 2, 3 and 16B a preferred method of the isolette assembly will be further described.

At step 1620, the stanchions are attached to the base panel, as previously described.

At step 1622, the top subassembly is positioned on the stanchions by inserting the stanchions into the appropriate stanchion pockets.

At step 1626, optionally, bi-directional gloves are attached to the access port, as previously described.

Referring to FIGS. 9, 10A, 10B, 11, 12A, 12B and 16C, a preferred method of the isolette assembly will be further described.

At step 1629, the brackets are connected to the bed rails.

At step 1630, the support frames are inserted in the appropriate support pockets.

At step 1632, the rear support pockets are secured by fixing the linear closures around the rear horizontal supports.

At step 1636, optionally, bi-directional gloves are fixed in the access ports.

Referring to FIGS. 13, 14 and 16D, a method of the isolette assembly will be further described.

At step 1640, the brackets are connected to the bed frame, as previously described.

At step 1642, the right and left support frames are positioned in the appropriate support pockets of the top subassembly.

At step 1644, optionally, bi-directional gloves are attached to and sealed within the access ports.

It will be appreciated by those skilled in the art that changes could be made to the embodiments described above without departing from the broad inventive concept thereof. It is understood, therefore, that this disclosure is not limited to the particular embodiments disclosed, but it is intended to cover modifications within the spirit and scope of the present disclosure as defined by the appended claims.

1. A medical isolette comprising:
a semi-rigid base panel;
a first set of vertical stanchions, attached to the semi-rigid base panel;
a second set of vertical stanchions, attached to the semi-rigid base panel;
a first flexible side panel sheet, forming a first set of vertical stanchion pockets, the first set of vertical stanchion pockets removably positioned on the first set of vertical stanchions;
a second flexible side panel sheet, forming a second set of vertical stanchion pockets, the second set of vertical stanchion pockets removably positioned on the second set of vertical stanchions;
a third flexible side panel sheet, sealed to the first flexible side panel sheet and the second flexible side panel sheet;
a flexible top panel sheet, sealed to the first flexible side panel sheet, the second flexible side panel sheet and the third flexible side panel sheet;
a flexible bottom panel sheet, sealed to the first flexible side panel sheet, the second flexible side panel sheet and the third flexible side panel sheet;
a flexible drape, sealed to the flexible top panel sheet, the first flexible side panel sheet and the second flexible side panel sheet;
a pressure intake, ductedly sealed to one of the group of the first flexible side panel sheet, the second flexible side panel sheet and the third flexible side panel sheet; and,
wherein application of a gas pressure to the pressure intake creates one of the group of a vacuum and a positive pressure to an interior of the medical isolette.

2. The medical isolette of claim 1 further comprising:
an access port, formed in one of the group of the first flexible side panel sheet, the second flexible side panel sheet and the third flexible side panel sheet.

3. The medical isolette of claim 2 further comprising:
a cover flap, fixed adjacent the access port; and,
a closure means, adjacent the cover flap, for sealing the cover flap against the access port.

4. The medical isolette of claim 2 wherein the access port further comprises:
a semi-rigid connection ring fixed in the access port;
a semi-rigid glove collar, removably fitted in the semi-rigid connection ring; and,
a sealed glove, sealed to the semi-rigid glove collar, and extending into an interior of the medical isolette.

5. The medical isolette of claim 2 wherein the access port further comprises:
a semi-rigid connection ring fixed in the access port; and,
a barrier cap, removably positioned in the semi-rigid connection ring.

6. The medical isolette of claim 2 further comprising:
an iris diaphragm, sealed within the access port.

7. The medical isolette of claim 2 further comprising:
an adhesive ring, positioned adjacent the access port;
a glove, having a glove sleeve; and,
wherein the glove sleeve is sealed to the adhesive ring.

8. The medical isolette of claim 2 further comprising:
a glove, having a glove sleeve and a thumb; and,
wherein the glove sleeve is integrated within the access port.

9. The medical isolette of claim 8 wherein the thumb is oriented upward relative to the medical isolette.

10. The medical isolette of claim 1 further comprising:
a component access panel, fixed in one of the group of the first flexible side panel sheet, the second flexible side panel sheet and the third flexible side panel sheet; and,
wherein a set of supply tubes accesses the isolette, through the component access panel, without loss of an appreciable internal pressure level of the medical isolette.

11. The medical isolette of claim 10 wherein the component access panel further comprises:
a semi-rigid connector block; and,
a set of double-ended connectors, ductedly positioned through the semi-rigid connector block.

12. The medical isolette of claim 10 wherein the component access panel further comprises:
a set of flexible seal portals; and,
a set of removable plugs in the set of flexible set of seal portals.

13. The medical isolette of claim 1 wherein:
the first set of vertical stanchions further comprises a first front stanchion and a first rear stanchion;
the second set of vertical stanchions further comprises a second front stanchion and a second rear stanchion;
wherein the first front stanchion and the second front stanchion are shorter than the first rear stanchion and the second rear stanchion; and,
whereby the flexible top panel sheet is positioned at a downward angle oriented toward a front of the medical isolette.

14. A medical isolette comprising:
a first longitudinal bracket;
a second longitudinal bracket, opposite the first longitudinal bracket;
a first forward facing perimeter support and a first rearward facing horizontal support, rigidly attached to the first longitudinal bracket;
a second forward facing perimeter support and a second rearward facing horizontal support, rigidly attached to the second longitudinal bracket;
a first flexible side panel sheet, forming a first forward facing perimeter support pocket and a first rearward facing horizontal support pocket;
the first forward facing perimeter support positioned in the first forward facing perimeter support pocket;
the first rearward facing horizontal support positioned in the first rearward facing horizontal support pocket;
a second flexible side panel sheet, forming a second forward facing perimeter support pocket and a second rearward facing horizontal support pocket;
the second forward facing perimeter support positioned in the second forward facing perimeter pocket;
the second rearward facing horizontal support positioned in the second rearward facing horizontal support pocket;
a third flexible side panel sheet, sealed to the first flexible side panel sheet and the second flexible side panel sheet;
a flexible top panel sheet, sealed to the first flexible side panel sheet, the second flexible side panel sheet, and the third flexible side panel sheet;
a flexible bottom panel sheet, sealed to the first flexible side panel sheet, the second flexible side panel sheet and the third flexible side panel sheet;
a drape sealed to the first flexible side panel sheet, the second flexible side panel sheet, and the flexible top panel sheet;
a pressure intake, ductedly connected to one of the group of the first flexible side panel sheet, the second flexible side panel sheet and the third flexible side panel sheet; and,
wherein application of a gas pressure to the pressure intake creates one of the group of a vacuum and a positive pressure to an interior of the medical isolette.

15. The medical isolette of claim 14 wherein the drape further comprises:
a distal edge; and,
a compressible seal, adjacent to the distal edge.

16. The medical isolette of claim 15 wherein the drape further comprises:
a securing strap, adjacent the compressible seal.

17. The medical isolette of claim 14 further comprising:
a first front access port, in the third flexible side panel sheet, between the first forward facing perimeter support and the second forward facing perimeter support.

18. The medical isolette of claim 17 further comprising:
a second front access port, adjacent the first front access port, between the first forward facing perimeter support and the second forward facing perimeter support.

19. The medical isolette of claim 18 further comprising:
a first bi-directional glove, sealed in the first front access port; and,
a second bi-direction glove, sealed in the second front access port.

20. The medical isolette of claim 19 wherein:
the first bi-directional glove has a first thumb sheath;
the second bi-directional glove has a second thumb sheath; and,
the first thumb sheath and the second thumb sheath are oriented upwards with respect to the medical isolette.

21. The medical isolette of claim 14 further comprising:
a first side access port, in the first flexible side panel sheet, centrally located under the first forward facing perimeter support.

22. The medical isolette of claim 21 further comprising:
a second side access port, in the second flexible side panel sheet, centrally located under the second forward facing perimeter support.

23. The medical isolette of claim 14 wherein the first rearward facing horizontal support pocket further comprises:
a first longitudinal resealable linear closure.

24. The medical isolette of claim 14 wherein the second rearward facing horizontal support pocket further comprises:
a second longitudinal resealable linear closure.

25. The medical isolette of claim 14 wherein the first longitudinal bracket further comprises:
a first c-channel cross section.

26. The medical isolette of claim 14 wherein the second longitudinal bracket further comprises:
a second c-channel cross section.

27. The medical isolette of claim 14 wherein the first forward facing perimeter support further comprises a first downward sloping top support rail;
wherein the second forward facing perimeter support further comprises a second downward sloping top support rail; and,
whereby the flexible top panel sheet is positioned at a downward angle.

28. A medical isolette comprising:
a first longitudinal bracket;
a second longitudinal bracket, opposite the first longitudinal bracket;
a first forward s-post and a first rearward s-post, rigidly attached to the first longitudinal bracket;
a second forward s-post and a second rearward s-post, rigidly attached to the second longitudinal bracket;
a first flexible side panel sheet, forming a first set of stanchion pockets, the first set of stanchion pockets removably positioned on the first forward s-post and the first rearward s-post;
a second flexible side panel sheet, forming a second set of stanchion pockets, the second set of stanchion pockets removably positioned on the second forward s-post and the second rearward s-post;
a third flexible side panel sheet, sealed to the first flexible side panel sheet and the second flexible side panel sheet;
a flexible bottom panel sheet, sealed to the first flexible side panel sheet, the second flexible side panel sheet and the third flexible side panel sheet;
a flexible top panel sheet, sealed to the first flexible side panel sheet, the second flexible side panel sheet and the third flexible side panel sheet;
a flexible drape, sealed to the flexible top panel sheet, the first flexible side panel sheet and the second flexible side panel sheet;
a pressure intake, ductedly sealed to one of the group of the first flexible side panel sheet, the second flexible side panel sheet and the third flexible side panel sheet; and,
wherein application of a gas pressure to the pressure intake creates one of the group of a vacuum and a positive pressure to an interior of the medical isolette.

29. The medical isolette of claim 28 further comprising:
an access port, formed in one of the group of the first flexible side panel sheet, the second flexible side panel sheet and the third flexible side panel sheet.

30. The medical isolette of claim 29 further comprising:
a cover flap, fixed adjacent the access port; and,
a closure means, adjacent the cover flap, for sealing the cover flap against the access port.

31. The medical isolette of claim 29 wherein the access port further comprises:
a semi-rigid connection ring fixed in the access port;
a semi-rigid glove collar, removably fitted in the semi-rigid connection ring; and,
a sealed glove, sealed to the semi-rigid glove collar, and extending into an interior of the medical isolette.

32. The medical isolette of claim 29 wherein the access port further comprises:
a semi-rigid connection ring fixed in the access port; and,
a barrier cap, removably positioned in the semi-rigid connection ring.

33. The medical isolette of claim 29 further comprising:
an iris diaphragm, sealed within the access port.

34. The medical isolette of claim 29 further comprising:
an adhesive ring, positioned adjacent the access port;
a glove, having a glove sleeve; and,
wherein the glove sleeve is sealed to the adhesive ring.

35. The medical isolette of claim 29 further comprising:
a glove, having a glove sleeve and a thumb; and,
wherein the glove sleeve is integrated within the access port.

36. The medical isolette of claim 35 wherein the thumb is oriented upward relative to the medical isolette.

37. The medical isolette of claim 28 further comprising:
a component access panel, fixed in one of the group of a flexible top panel sheet, a first flexible side panel sheet, a second flexible side panel sheet and a third flexible side panel sheet; and,
wherein a set of supply tubes accesses the isolette, through the component access panel, without loss of an appreciable internal pressure level.

38. The medical isolette of claim 37 wherein the component access panel further comprises:
a semi-rigid connector block; and,
a set of double-ended connectors, ductedly positioned through the semi-rigid connector block.

39. The medical isolette of claim 37 wherein the component access panel further comprises:
a set of flexible seal portals; and,
a set of removable plugs in the set of flexible set of seal portals.

40. The medical isolette of claim 28 wherein:
the first forward s-post and the second forward s-post are shorter than the first rearward s-post and the second rearward s-post; and,
whereby the flexible top panel sheet is positioned at a downward sloping angle.

41. A method of use of a medical isolette comprising:
providing a semi-rigid base panel;
providing a first set of vertical stanchions, attached to the semi-rigid base panel;
providing a second set of vertical stanchions, attached to the semi-rigid base panel;
providing a first flexible side panel sheet, forming a first set of vertical stanchion pockets, the first set of vertical stanchion pockets removably positioned on the first set of vertical stanchions;
providing a second flexible side panel sheet, forming a second set of vertical stanchion pockets, the second set of vertical stanchion pockets removably positioned on the second set of vertical stanchions;
providing a third flexible side panel sheet, connected to the first flexible side panel sheet and the second flexible side panel sheet;
providing a flexible top panel sheet, connected to the first flexible side panel sheet, the second flexible side panel sheet and the third flexible side panel sheet;
providing a flexible bottom panel sheet, connected to the first flexible side panel sheet, the second flexible side panel sheet and the third flexible side panel sheet;
providing a flexible drape, connected to the flexible top panel sheet, the first flexible side panel sheet and the second flexible side panel sheet;
providing a pressure intake, ductedly connected to one of the group of the first flexible side panel sheet, the second flexible side panel sheet and the third flexible side panel sheet;
providing an access port, formed in one of the group of the first flexible side panel sheet, the second flexible side panel sheet and the third flexible side panel sheet;
extending the drape;
securing the drape;
connecting a pressurization device to the pressure intake;
accessing an interior of the medical isolette through the access port; and,
applying a gas pressure to the medical isolette to the pressure intake with the pressurization device.

42. The method of claim 41 further comprising:
attaching the first set of vertical stanchions and the second set of vertical stanchions to the semi-rigid base panel;
positioning the first set of vertical stanchion pockets on the first set of vertical stanchions;
positioning the second set of vertical stanchion pockets on the second set of vertical stanchions; and,
sealing the first flexible side panel sheet, the second flexible side panel sheet and the third flexible side panel sheet to a perimeter of the semi-rigid base panel.

43. The method of claim 41 further comprising:
providing an access port in one of the first flexible side panel sheet, the second flexible side panel sheet and the third flexible side panel sheet; and,
sealing a bi-direction glove in the access port.

44. The method of claim 43 wherein the step of providing an access port further comprises:
cutting a hole in one of the first flexible side panel sheet, the second flexible side panel sheet and the third flexible side panel sheet.

45. A method of use of a medical isolette comprising:
providing a first longitudinal bracket;
providing a second longitudinal bracket, opposite the first longitudinal bracket;
providing a first forward facing perimeter support and a first rearward facing horizontal support, rigidly attached to the first longitudinal bracket;
providing a second forward facing perimeter support and a second rearward facing horizontal support, rigidly attached to the second longitudinal bracket;
providing a first flexible side panel sheet, forming a first forward facing perimeter support pocket and a first rearward facing horizontal support pocket;
positioning the first forward facing perimeter support in the first forward facing perimeter support pocket;
positioning the first rearward facing horizontal support in the first rearward facing horizontal support pocket;
providing a second flexible side panel sheet, forming a second forward facing perimeter support pocket and a second rearward facing horizontal support pocket;
positioning the second forward facing perimeter support in the second forward facing perimeter pocket;
positioning the second rearward facing horizontal support in the second rearward facing horizontal support pocket;
providing a third flexible side panel sheet, attached to the first flexible side panel sheet and the second flexible side panel sheet;
providing a flexible bottom panel sheet, connected to the first flexible side panel sheet, the second flexible side panel sheet and the third flexible side panel sheet;
providing a flexible top panel sheet, attached to the first flexible side panel sheet, the second flexible side panel sheet, and the third flexible side panel sheet;
providing a drape attached to the first flexible side panel sheet, the second flexible side panel sheet, and the flexible top panel sheet;
providing a pressure intake, ductedly connected to one of the group of the first flexible side panel sheet, the second flexible side panel sheet and the third flexible side panel sheet;
extending the drape;
securing the drape;
connecting a pressurization device to the pressure intake; and,
applying gas pressure to the medical isolette through the pressurization intake with the pressurization device.

46. The method of claim 45 further comprising:
inserting the first forward facing perimeter support into the first forward facing perimeter support pocket;
inserting the second forward facing perimeter support into the second forward facing perimeter support pocket;
inserting the first rearward facing horizontal support into the first rearward horizontal support pocket; and,
inserting the second rearward facing horizontal support into the second rearward horizontal support pocket.

47. The method of claim 46 further comprising:
attaching the first longitudinal bracket and the second longitudinal bracket to a bedframe.

48. The method of claim 46 further comprising:
providing an access port in one of the first flexible side panel sheet, the second flexible side panel sheet and the third flexible side panel sheet; and,
positioning a bi-directional glove in the access port.

49. The method of claim 43 wherein the step of providing an access port further comprises:
cutting a hole in one of the first flexible side panel sheet, the second flexible side panel sheet and the third flexible side panel sheet.

50. A method of use of a medical isolette comprising:
providing a first longitudinal bracket;
providing a second longitudinal bracket, opposite the first longitudinal bracket;
providing a first forward s-post and a first rearward s-post, rigidly attached to the first longitudinal bracket;
providing a second forward s-post and a second rearward s-post, rigidly attached to the second longitudinal bracket;
providing a first flexible side panel sheet, forming a first set of stanchion pockets;
removably positioning the first set of stanchion pockets removably positioned on the first forward s-post and the first rearward s-post;
providing a second flexible side panel sheet, forming a second set of stanchion pockets;
removably positioning the second set of stanchion pockets on the second forward s-post and the second rearward s-post;
providing a third flexible side panel sheet, connected to the first flexible side panel sheet and the second flexible side panel sheet;
providing a flexible top panel sheet, connected to the first flexible side panel sheet, the second flexible side panel sheet and the third flexible side panel sheet;
providing a flexible bottom panel sheet, connected to the first flexible side panel sheet, the second flexible side panel sheet and the third flexible side panel sheet;
providing a flexible drape, connected to the flexible top panel sheet, the first flexible side panel sheet and the second flexible side panel sheet;
providing a pressure intake, ductedly connected to one of the group of the first flexible side panel sheet, the second flexible side panel sheet and the third flexible side panel sheet; and,
providing an access port, formed in one of the group of the first flexible side panel sheet, the second flexible side panel sheet and the third flexible side panel sheet.

51. The method of claim 50 further comprising:
inserting the first forward s-post and the first rearward s-post into the first set of stanchion pockets.

52. The method of claim 50 further comprising:
inserting the second forward s-post and the second rearward s-post into the second set of stanchion pockets.

53. The method of claim 46 further comprising:
attaching the first longitudinal bracket and the second longitudinal bracket to a bedframe.

54. The method of claim 46 further comprising:
providing an access port in one of the first flexible side panel sheet, the second flexible side panel sheet and the third flexible side panel sheet; and,
positioning a bi-directional glove in the access port.

55. The method of claim 43 wherein the step of providing an access port further comprises:
cutting a hole in one of the first flexible side panel sheet, the second flexible side panel sheet and the third flexible side panel sheet.
